(19) United States
(12) Patent Application Publication
(10) Pub. No.: US 2011/0105365 A1
(43) Pub. Date: May 5, 2011

(54) PRO-EPIL EXPRESSION LEVEL IN A BIOLOGICAL SAMPLE AS TESTICULAR CANCER BIOMARKER, PARTICULARLY IN COMBINATION WITH THE HCBETA AND AFP BIOMARKERS

(75) Inventors: Dominique Bellet, Boulogne (FR); Alain Pecking, Colombes (FR); Sophie Richon, L'Isle Adam (FR); Felice Petraglia, Siena (IT)

(73) Assignees: Universite Paris Descartes, Paris (FR); Centre National De La Recherche Scientifique (CNRS), Paris (FR); Universita Degli Studi Di Siena, Siena (IT); Institut Curie (formerly Centre Rene Huguenin), Paris (FR); Institut Gustave Roussy, Villejuif (FR)

(21) Appl. No.: 12/989,999

(22) PCT Filed: Apr. 28, 2009
(86) PCT No.: PCT/EP09/55100

§ 371 Date: Jan. 13, 2011

Related U.S. Application Data
(60) Provisional application No. 61/048,412, filed on Apr. 28, 2008.

(51) Int. Cl. C12Q 1/68; G01N 33/68; C40B 40/06; C40B 40/10

(52) U.S. Cl. 506/16; 435/6; 435/7.1; 436/501; 435/7.92; 506/18

(57) ABSTRACT

The present invention is directed to the use of the expression level of the pro-EPIL gene as a biomarker for the diagnosing of testicular cancer, particularly a testicular germ cell tumor. The invention also relates to an in vitro method for detecting and/or classifying a testicular cancer in a subject comprising a step of determining the expression level of the gene encoding the pro-EPIL peptide in a biological, particularly in combination with the determination of the beta subunit HCGβ and the human alpha-fetoprotein AFP. The invention is also directed to a kit or solid support comprising nucleic acids or antibodies capable of determining the presence or the expression level of these three biological markers.

The present invention is directed to the use of the expression level of the pro-EPIL gene as a biomarker for the diagnosing of testicular cancer, particularly a testicular germ cell tumor. The invention also relates to an in vitro method for detecting and/or classifying a testicular cancer in a subject comprising a step of determining the expression level of the gene encoding the pro-EPIL peptide in a biological, particularly in combination with the determination of the beta subunit HCGβ and the human alpha-fetoprotein AFP. The invention is also directed to a kit or solid support comprising nucleic acids or antibodies capable of determining the presence or the expression level of these three biological markers.

Management of patients with testicular cancers is a success in modern oncology, since more than 90 percent of patients with newly diagnosed germ-cell tumors are cured and testicular cancer mortality rates have fallen by about 70% in the USA and western Europe since the 1970s.¹⁻³ This success is due to two major improvements in management of these neoplasms: the introduction of cisplatin-containing combination chemotherapy that dramatically improved the cure rate, and simultaneous introduction of serum tumor markers, namely human chorionic gonadotropin (HCG), its free beta subunit (HCGβ) and alpha-fetoprotein (AFP). These tumor markers help in diagnosis and play an important role in assessment of response to treatment and in monitoring remission.⁴ Lactate dehydrogenase (LDH) is also a germ cell tumor product, but it is a less specific tumor marker.

One factor that affects prognosis is the delay in diagnosing testicular tumors, which impacts the stage and therefore the prognosis. During the last two decades, there has been a trend over time towards reduction of diagnostic delay.⁵ However, recent results of a large population-based study show that long diagnostic delay is associated with lower survival for patients with non-seminomatous tumors.⁶ Moreover, the worldwide incidence of testicular cancer has more than doubled over the past 40 years. Recently, its incidence has been rising in nearly all industrialized countries.⁷ Despite improvements that occurred 30 years ago, the decline in mortality rates has begun to slow in the past few years in Europe, the USA and Japan, indicating the possible approach of an asymptote in testis cancer mortality.³ Patients with advanced disease must be treated with chemotherapy regimens that have substantial toxicity, and some of these patients fail to respond to such treatment.

In this context, it would be worthwhile to further reduce diagnostic delay and to continue to improve treatment modalities for testicular tumors so as to reduce the extent of such therapy and the burden of their toxicity. Novel biomarkers might contribute to achieving such a goal.

This is the object of the present invention.

The inventors have demonstrated that pro-EPIL peptide, or specific fragments thereof, can be used as a tumor biomarker for detecting and/or classifying a testicular cancer in a subject, particularly as a class of testicular germ cell tumor.

The EPIL peptide is encoded by the insulin-like 4 gene (INSL4). This gene was identified by screening a cDNA library of first-trimester human placenta. INSL4 is highly expressed in early placenta and, with the exception of faint expression in normal uterine tissues, expression of INSL4 transcripts was not detected in any other normal tissue tested thus far.⁸ The early placenta insulin-like peptide (EPIL) encoded by the insulin-like 4 gene is a member of the insulin-related gene family comprising insulin, relaxin (RLX), insulin-like growth factors 1 and II (IGFI I and IGF II), Leydig insulin-like peptide (LEY I-L) encoded by the INSL3 gene and peptides encoded by INSL5 and INSL6 genes.

EPIL is a 139-amino-acid polypeptide which is synthesized as a preprohormone characterized by a signal peptide, a B-chain, a connecting C-peptide and a terminal A-chain (FIG. 1). In placenta, it was found that trophoblast cells translate INSL4 mRNAs into immunoreactive pro-EPIL peptides comprising the B-, C- and A-chains.⁹ Initially, pro-EPIL peptide was detected in amniotic fluid and maternal serum during normal pregnancy, and the excretion pattern of pro-EPIL was similar to that of HCGβ, suggesting common regulation pathways.¹⁰

The inventors have demonstrated that pro-EPIL peptide (PEP) is expressed and secreted by testicular germ cell tumors (TGCTs). PEP expression was investigated in patients with testicular cancers at the cellular and serum levels, enabling to show that PEP is a novel biomarker which provides additional information in comparison to that provided by HCGβ and AFP.

Thus, the present invention is directed to an in vitro method for detecting and/or classifying a testicular cancer in a subject, comprising the step of determining the expression level of the gene encoding the pro-EPIL peptide in a biological sample isolated from said subject wherein overexpression of said gene encoding the pro-EPIL peptide is indicative of the presence of a testicular cancer and/or class of testicular germ cell tumor.

In a preferred embodiment, the method according to the invention further comprises a step of determining the expression level of at least one gene selected from the group of gene consisting of the genes encoding the human gonadotropin HCG, its beta subunit HCGβ and the human alpha-fetoprotein AFP in a biological sample isolated from said subject wherein overexpression of at least one of said gene encoding the encoding the HCG, its beta subunit HCGβ or the human AFP is indicative of the presence of a testicular cancer and/or class of testicular germ cell tumor.

In a more preferred embodiment, said testicular cancer and/or class of testicular germ cell tumor is seminatous or non-seminatous.

The methods for determining the expression level of the genes encoding the human pro-EPIL, the human HCG, or its subunit HCGβ, and the human AFP in a biological sample isolated from said subject are well known by the skill person.

In a more preferred embodiment, in the method according to the invention, the presence of an overexpression of the gene encoding the pro-EPIL peptide, an overexpression of the gene encoding the HCGβ and an overexpression of the gene encoding the alpha-fetoprotein AFP in a biological sample isolated from said is indicative of the presence of a testicular cancer and/or class of testicular germ cell tumor of non-seminatous form.

In another aspect, the present invention is directed to a method for identifying a compound candidate for a pharmacological agent useful in the treatment of testicular cancer, particularly a testicular germ cell tumor, comprising the step of:

a) contacting a non-human mammal subject presenting a testicular cancer, particularly a testicular germ cell tumor, with a candidate pharmacological agent;
b) determining the expression level of the gene encoding the pro-EPIL peptide in a biological sample isolated from said subject;
c) optionally, further determining the expression level of at least one gene selected from the group of gene consisting of the genes encoding the HCG, its subunit HCGβ and the human AFP in a biological sample isolated from said subject,
wherein a decrease in the test amount of expression of the gene encoding the pro-EPIL peptide, and optionally, a decrease of the expression of the gene determined in step c), indicates that the candidate pharmacological agent is a potential compound for a pharmacological agent useful in the treatment of testicular cancer, particularly a testicular germ cell tumor.

In another aspect, the present invention is directed to a method for evaluating the effect in a subject of a treatment for testicular cancer, particularly a testicular germ cell tumor, comprising the step of:

a) determining the expression level of the gene encoding the pro-EPIL peptide in a biological sample isolated from said subject;
b) determining a second expression level of the gene encoding the pro-EPIL peptide in a biological sample isolated from said subject;
c) comparing the first and second amounts of expression level of the gene encoding the pro-EPIL peptide in a biological sample isolated from said subject;
wherein a decrease of the expression level of the gene encoding the pro-EPIL peptide in the second biological sample isolated from said subject indicates the regression of said testicular cancer, particularly a testicular germ cell tumor.

In the methods according to the present invention as defined above, it is preferred that the expression level is determined by detecting the presence, absence or level of mRNA transcribed from said INSL4 gene or of pro-EPIL peptide encoded by said gene, or specific peptidic fragment thereof. The detection of the presence, absence or level of pro-EPIL peptide encoded by said gene, or specific peptide fragment thereof is more preferred.

The amino-acid and mRNA sequence of the human Early placental insulin-like protein (INSL4 or EPIL, see GenPep accession number NP_{—}002186) is:

Complete pro-EPIL amino acids sequence (SEQ ID NO: 1)

```
    1 MASLFRSYLP AIWLLLSQLL RESLAAELRG CGPRFGKHLL SYCPMPEKTF
  51 TTTPGGWLLE SGRPKEMVST SNNKDGQALG TTSEFIPNLS PELKKPLSEG
101 QPSLKKIILS RKKRSGRHRF DPFCCEVICD DGTSVKLCT
```

aa1-22: signal peptide (SEQ ID NO: 2)
aa23-52: “B chain” (SEQ ID NO: 3)
aa59-108: “C chain (SEQ ID NO: 4)
aa115-139: “A chain” (SEQ ID NO: 5).

Insulin-like 4 (placenta) (INSL4), mRNA (see Genbank accession number NM_{—}002195, SEQ ID NO: 6

```
    1 agtctggagc ccagaaggga cacaccagca cagtctggta ggctacagca gcaagtctct
  61 aaagaaaggc tgagaacacc cagaacagga gagttcaggt ccaggatggc cagcctgttc
121 cggtcctatc tgccagcaat ctggctgctg ctgagccaac tccttagaga aagcctagca
181 gcagagctga ggggatgtgg tccccgattt ggaaaacact tgctgtcata ttgccccatg
241 cctgagaaga cattcaccac caccccagga gggtggctgc tggaatctgg acgtcccaaa
301 gaaatggtgt caacctccaa caacaaagat ggacaagcct taggtacgac atcagaattc
361 attcctaatt tgtcaccaga gctgaagaaa ccactgtctg aagggcagcc atcattgaag
421 aaaataatac tttcccgcaa aaagagaagt ggacgtcaca gatttgatcc attctgttgt
481 gaagtaattt gtgacgatgg aacttcagtt aaattatgta catagtagag taatcatgga
541 ctggacatct catccattct catatgtatt ctcaatgaca aattcactga tgcccaatta
601 aatgattgct gtttaaa
```

nt106-522: pro-EPIL coding sequence (SEQ ID NO: 7)
nt106-171: signal peptide (SEQ ID NO: 8)
nt172-261: “B chain” (SEQ ID NO: 9)
nt280-429: “C chain (SEQ ID NO: 10)
nt448-522: “A chain >>(SEQ ID NO: 11).

INSL4 gene encodes a precursor that undergoes post-translational cleavage to produce 3 polypeptide chains, A-C, that form tertiary structures composed of either all three chains, or just the A and B chains (Chassin, D., Laurent, A., Janneau, J. L., Berger, R. and Bellet, D., Genomics 29 (2), 465-470 (1995)).

It shall be understood that the term “specific peptide fragment” designates in particular a fragment of an amino acid sequence of a polypeptide having at least one of the functional characteristics or properties of the complete polypeptide, notably in that it is capable of being recognized by a specific antibody and/or that the expression level of such a specific peptide fragment is correlated to expression level of the complete or partial pro-EPIL expressed.

It is understood that the term “specific peptide fragment” designates particularly a polypeptide including a minimum of 9 amino acids, preferably 10, 11 or 12 amino acids, and most preferably 15, 20 or 25 amino acids of the sequence SEQ ID No: 1, preferably this fragment contains a fragment of at least the chain A, B or C of the human pro-EPIL.

Specific anti-pro-EPIL monoclonal or polyclonal antibodies are available to the skilled man. An isolated pro-EPIL, or a specific fragment thereof, can be used as an immunogen to generate antibodies that bind such protein using standard techniques for polyclonal and monoclonal antibody preparation. It may be also possible to use any fragment of these protein which contains at least one antigenic determinant may be used to generate these specific antibodies.

A protein immunogen typically is used to prepare antibodies by immunizing a suitable subject, (e.g., rabbit, goat, mouse or other mammal) with the immunogen. An appropriate immunogenic preparation can contain said pro-EPIL polypeptide, or fragment thereof, and further can include an adjuvant, such as Freund's complete or incomplete adjuvant, or similar immuno-stimulatory agent.

Thus, antibody for use in accordance with the invention include either polyclonal, monoclonal chimeric or humanized antibodies. antibodies able to selectively bind, or which selectively bind to an epitope-containing a pro-EPIL polypeptide comprising a contiguous span of at least 9 to 10 amino acids of a pro-EPIL fragment, particularly of a fragment of at least the chain A, B or C of the human pro-EPIL.

A preferred agent for detecting and quantifying mRNA or cDNA encoding the human pro-EPIL is a labeled nucleic acid probe or primers able to hybridize this mRNA or cDNA. The nucleic acid probe can be an oligonucleotide of at least 10, 15, 30, 50 or 100 nucleotides in length and sufficient to specifically hybridize under stringent conditions to the mRNA or cDNA. The nucleic acid primer can be an oligonucleotide of at least 10, 15 or 20 nucleotides in length and sufficient to specifically hybridize under stringent conditions to the mRNA or cDNA, or complementary sequence thereof.

A preferred agent for detecting and quantifying the human pro-EPIL, is an antibody able to bind specifically to this protein, preferably an antibody with a detectable label. Antibodies can be polyclonal, or more preferably, monoclonal. As used herein, the term encompasses not only intact polyclonal or monoclonal antibodies, but also fragments thereof (such as Fab, Fab′, F(ab′)2, Fv), single chain (ScFv), mutants thereof, fusion proteins comprising an antibody portion, humanized antibodies, chimeric antibodies, diabodies linear antibodies, single chain antibodies, multispecific antibodies (e.g., bispecific antibodies) and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

For example, in vitro techniques for detection of mRNA include Northern hybridizations and in situ hybridizations. In vitro techniques for detection of the candidate protein include enzyme linked immunosorbent assays (ELISAs), Western blots, immunoprecipitations and immunofluorescence. In vitro techniques for detection of candidate cDNA include Southern hybridizations.

When the invention encompasses kits for quantifying the level of the human pro-EPIL polypeptide or specific fragment thereof, the kit can comprise a labeled compound or agent capable of quantifying this polypeptide. Said agents can be packaged in a suitable container. The kit can further comprise instructions for using the kit to quantify the level of the human pro-EPIL or of the human pro-EPIL transcript.

In certain embodiments of the method of the present invention, the determination of the human pro-EPIL transcripts involves the use of a probe/primer in a polymerase chain reaction (PCR), such as anchor PCR or RACE PCR, or, alternatively, in a ligation chain reaction (LCR) (see, e.g., Landegran et al., 1988, Science 241:23-1080; and Nakazawa et al., 1994, Proc. Natl. Acad. Sci. USA, 91:360-364), or alternatively quantitative real time RT-PCR This method can include the steps of collecting a sample of cells from a patient, isolating nucleic acid (e.g. mRNA) from the cells of the sample, optionally transforming mRNA into corresponding cDNA, contacting the nucleic acid sample with one or more primers which specifically hybridize to the pro-EPIL mRNA or their corresponding cDNA under conditions such that hybridization and amplification of the pro-EPIL mRNA or cDNA occurs, and quantifying the presence of the amplification products. It is anticipated that PCR and/or LCR may be desirable to use as an amplification step in conjunction with any of the techniques used for quantifying nucleic acid detecting.

The methods described herein may be performed, for example, by utilizing pre-packaged diagnostic kits comprising at least one probe nucleic acid or set of primer or antibody reagent described herein, which may be conveniently used, e.g., in clinical settings to follow-up or diagnose patients.

In another preferred embodiment, the pro-EPIL peptide, or specific peptide fragment thereof, is detected or quantified by western blot analysis, chromatography, immunoassay or immunohistochemistry.

In a more preferred embodiment, said pro-EPIL peptide, is detected or quantified by ELISA immunoassay or radioimmunoassay.

In the method of the present invention, it is preferred that the biological sample obtained from the subject is selected from the group comprising whole blood, blood serum or plasma, tumor biopsy or combinations thereof, blood serum or plasma are more preferred.

In another aspect, the present invention is directed to a kit comprising:

a) an antibody directed specifically against the pro-EPIL peptide; and
b) an antibody directed specifically against at least one of the polypeptide selected from the group consisting of the HCG, its subunit HCGβ and the human AFP polypeptide, preferably:
a) an antibody directed specifically against the pro-EPIL peptide;
b) an antibody directed specifically against the HCG or its subunit HCGβ polypeptide; and
c) an antibody directed specifically against the human AFP polypeptide.

The present invention also relates to a kit comprising:

a) a set of primers capable of amplifying specifically the pro-EPIL RNA or cDNA; and
b) a set of primers capable of amplifying specifically the RNA or cDNA from the group consisting of the human HCG, its subunit HCGβ and the human alpha-fetoprotein AFP RNA or cDNA,
preferably:
a) a set of primers capable of amplifying specifically the pro-EPIL RNA or cDNA;
b) a set of primers capable of amplifying specifically the HCG or its subunit HCGβ RNA or cDNA; and
c) a set of primers capable of amplifying specifically the human AFP RNA or cDNA.

The present invention also relates to a kit comprising:

a) a nucleic probe capable of hybridising specifically with the pro-EPIL RNA; and
b) a nucleic probe capable of hybridising specifically with at least one of the RNA selected from the group consisting of the human gonadotropin HCG, its beta subunit HCGβ and the human alpha-fetoprotein AFP RNA,
preferably:
a) a nucleic probe capable of hybridising specifically with the pro-EPIL RNA;
b) a nucleic probe capable of hybridising specifically with the HCG or its subunit HCGβ RNA; and
c) a nucleic probe capable of hybridising specifically with the human AFP RNA.

In the methods or the kits according to the present invention, the antibodies or the probe can be labeled when it is necessary.

Are also comprised in the present invention the kits of the present invention which are suitable for performing the method for detecting and/or classifying a testicular cancer in a subject, to identifying compound of interest or to control the efficiency of an anti-cancer treatment according to the above claimed invention.

One or more reagents necessary to the detection or the quantification of the biomarker may be immobilized onto solid support, such as biochips to form a two-dimension array, for example, a 9 mm×9 mm array, 12 mm×12 mm array, and 15 mm×15 mm array. One or more arrays may be arranged on one biochip, and one or more samples can be tested using one biochip. In some embodiments, the solid support of the biochip comprises a surface selected from the group consisting of a ceramic, a glass, a silica, a quartz, a nylon, a plastic, a polystyrene, a nitrocellulose, and a metal. This type of support and method using bio chip (protein or nucleic acid biochip) are well known by the skilled man to perform diagnosing tests.

Thus, are also claimed in the present invention a solid-phase nucleic acid molecule array or a solid support comprising:

a) a nucleic acid molecule capable of hybridising specifically with the pro-EPIL RNA; and
b) a nucleic acid molecule capable of hybridising specifically with at least one of the RNA selected from the group consisting of the human gonadotropin HCG, its beta subunit HCGβ and the human alpha-fetoprotein AFP RNA, fixed to a solid substrate,
preferably solid-phase nucleic acid molecule array or solid support comprising:
a) an antibody directed specifically against the pro-EPIL peptide; and
b) an antibody directed specifically against at least one of the polypeptide selected from the group consisting of the HCG, its subunit HCGβ and the human alpha-fetoprotein AFP polypeptide,
fixed to a solid substrate.

Finally, the present invention is directed to the use of the expression of the pro-EPIL gene as a biomarker, preferably as a serum (or plasma) or cellular biomarker, for the diagnosing of testicular cancer, particularly of a testicular germ cell tumor.

It is to be understood that while the invention has been described in conjunction with the above embodiments, that the foregoing description and the following examples are intended to illustrate and not limit the scope of the invention. Other aspects, advantages and modifications within the scope of the invention will be apparent to those skilled in the art to which the invention pertains.

### LEGENDS OF THE FIGURES

FIG. 1: Schematic representation of the primary structure of pro-EPIL peptide and localization of antibody binding sites recognized by monoclonal antibodies EPIL15 and EPIL02. Amino acid residues are indicated by one-letter code.

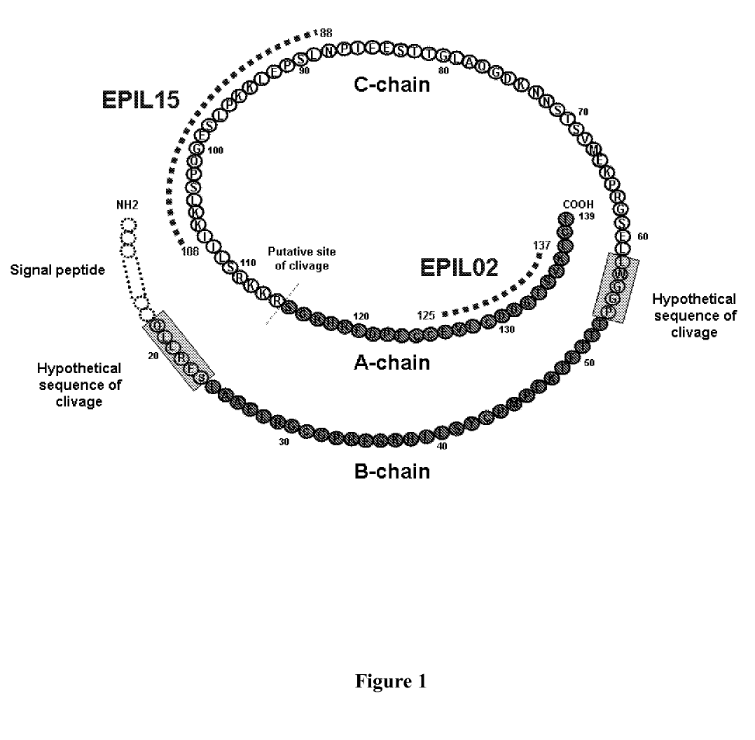

FIG. 2: Serum levels of PEP in healthy male subjects and in patients with seminomatous and non-seminomatous testicular germ-cell tumors. O, 10 cases; •, 1 case.

FIGS. 3A-3B: Representative examples of serum levels of PEP and HCGβ in two patients with non-seminomatous testicular germ-cell tumors: patient with a mixed tumor composed of embryonal carcinoma and seminoma. (A) Patient with a mixed tumor composed of choriocarcinoma, yolk sac tumor, teratoma and seminoma.

FIGS. 4A-4F: Immunohistochemistry staining of PEP (FIGS. 4A, 4C, 4E) and HCGβ (FIGS; 4B, 4D, 4F) in a mixed tumor composed of embryonal carcinoma, teratoma and choriocarcinoma (A, B) and in a mixed tumor composed of embryonal carcinoma, yolk sac tumor and seminoma with syncytiotrophoblastic cells (FIGS. 4C to 4F). Photos were taken at either original 10× (FIGS. 4A to 4D) or original 20× (FIGS. 4E, 4F) magnification.

### EXAMPLES

### 1) Patients and Methods

### a) Study Population and Sample Collection

All serum samples were selected from our institutional review-board-approved repository. The samples were all collected, processed and stored at −20° C. in a similar fashion. Serum samples from 52 patients with testicular germ-cell cancer and followed for up to 10 years, and from 104 healthy male subjects, were studied after receiving their informed consent. Patient demographics and histology are presented in Table 1.

TABLE 1
Patient demographics and histology of testicular germ-cell tumors

| | | | Patients |
|---|---|---|---|
| | | | Number |
| | Patients | | |
| Demographics | Age in years | 34.8 + 10.9 | 52 |
| | range | 17-69 | |
| | Serum level measurement | | |
| | | before orchidectomy | 9 |
| | | after orchidectomy | 43 |
| | Delay between orchidectomy | | |
| | and serum measurement | | |
| | Days | 59 + 66.2 | |
| | Range | 1-303 | |
| | Tumors | | |
| Histology | Seminomatous | | 25 |
| | Seminoma | | 21 |
| | Seminoma with | | 4 |
| | syncytiotrophoblastic | | |
| | cells | | |
| | Non-seminomatous | | 27 |
| | Pure forms | | 5 |
| | Embryonal carcinoma | | 4 |
| | Yolk sac | | 1 |
| | Mixed forms | | 22 |
| | Embryonal carcinoma + | | 5 |
| | seminoma | | |
| | Embryonal carcinoma + | | 2 |
| | yolk sac | | |
| | Embryonal carcinoma + | | 1 |
| | teratoma | | |
| | Teratoma + seminoma | | 1 |
| | Choriocarcinoma + | | 1 |
| | seminoma | | |
| | Other (more than two | | 12 |
| | histological types) | | |

Clinical data, including stage at diagnosis, histology, treatment modalities and outcome, were available for each patient whose serum was used in the study. Moreover, tissue specimens from two patients with testicular cancer were selected from the established tumor library of our comprehensive cancer Center, in accordance with protocols that had been approved by the local ethical committee. Tissue specimens were fixed in 10% neutral-buffered formalin and paraffin-embedded using standard procedures. Serial sections (5 μm) were cut for histology and immunohistochemistry.

PEP enzyme-linked immunosorbent assay and immunoassays for detection of HCGβ and AFP.

PEP serum levels were measured in sera using an ELISA based on monoclonal antibody (mAb) EPIL15 (kindly supplied by Prof. Bidart, Institut Gustave-Roussy, Villejuif, France) directed to the EPIL C-chain 98-108 region as previously described.¹¹ This antibody serves as capture antibody on a solid phase support, while biotinylated mAb EPIL02 (kindly supplied by Prof. Bidart, Institut Gustave-Roussy, Villejuif, France) directed to the EPIL A-chain 125-137 region is used as tracer. The standard curve was constructed with a peptide spanning the 76-139 portion of pro-EPIL used at increasing concentrations ranging from 0.7 ng/mL to 100 ng/mL. Linearity in a range of 0.7 ng/mL to 100 ng/mL was consistently shown in between and within-run assays. The coefficients of variation interassay (n=10) and intra-assay (n=33) were 12% and 13.3%, respectively at 6.6 ng/mL and 6% and 7.5%, respectively at 50 ng/mL. The sensitivity of this ELISA is 1 ng/mL.

A commercial IRMA (ELSA-FβHCG, CIS bio international, Gif-sur-Yvette, France) based on highly specific monoclonal antibody mAb FBT11 was used to measure HCGβ. The immunoassay for HCGβ displays a sensitivity of 100 pg/mL.¹² AFP serum levels were measured by a commercial immunoassay (BRAHMS-AFP Kryptor, Hennigsdorf, Germany) based on highly specific mAb AF01.¹³ This assay displays a sensitivity of 0.23 ng/mL.

### b) Histology and Immunohistochemistry

Histology of each specimen was determined by visual examination of tissue sections stained with hematoxylin, eosin and safran. Immunostaining was performed using an automated immunostainer (Dako Autostainer) and a streptavidine biotine peroxidase method (Dako Real™ Detection System LSAB+, Dako SAS, Trappes, France) with mAb EPIL15 directed to PEP, polyclonal antibody directed to HCG (Dako SAS, Trappes, France) and polyclonal antibody directed to AFP (Dako SAS, Trappes, France).

### c) Statistical Analysis

Statistical analyses were performed using GraphPad Prism version 4.0. Correlations were studied with non-parametric (Spearman) tests.

### 2) Results

### a) Serum Levels of PEP, HCGβ and AFP at First Determination

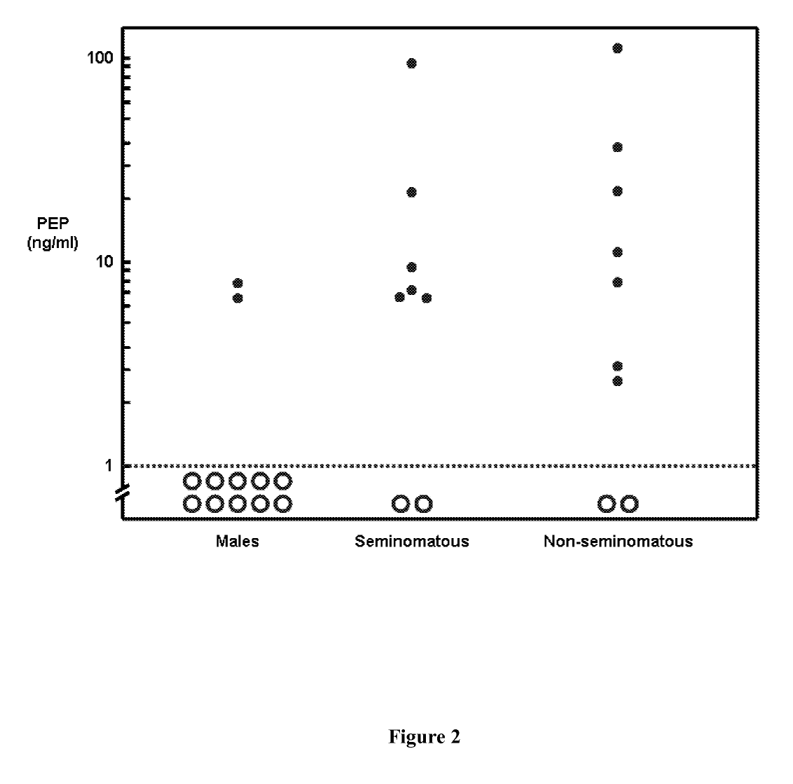

Serum levels of PEP, HCGβ and AFP were measured in 25 patients with seminomatous testicular germ-cell tumors and in 27 patients with non-seminomatous tumors (Table 1). Serum samples were collected before orchidectomy in 9 patients and after orchidectomy in 43 patients. In parallel, serum levels of PEP were measured in 104 healthy male subjects. PEP serum values are shown in FIG. 2. In healthy subjects, only 2 out of 104 (1.9%) had serum values higher than 1 ng/mL, while 13 out 52 patients (25%) displayed serum PEP levels higher than 1 ng/mL. In patients with seminomatous and non-seminomatous germ cell tumors, PEP was present in 24% and 25% of serum samples at first determination, respectively. In this series of 52 patients, serum levels of HCGβ was detected in 17 patients (32.6%) and AFP was elevated in the sera of 12 patients (23%). Data analysis with non-parametric (Spearman) tests showed that PEP serum values were not correlated with serum levels of either HCGβ (r=0.0076) or AFP (r=−0.0548). Indeed, one and/or two of the latter biomarkers were present in 44.2% of patients, while PEP, HCGβ and/or AFP were detected in the sera of 59.6% of patients with seminomatous or non-seminomatous testicular cancers (Table 2).

TABLE 2
Patients with elevated serum levels of PEP (>1 ng/ml),
HCGβ (>0.1 ng/ml) and/or AFP (>10 ng/ml)

| | | Serum biomarker(s) | | | | |
|---|---|---|---|---|---|---|
| | | | | | | PEP |
| | | | | | Free | or |
| | | | | | HCGβ | HCGβ |
| | Patient | | | | or | or |
| Histology | Number | PEP | HCGβ | AFP | AFP | AFP |
| Seminomatous | 25 | 6 | 9 | 0 | 10 | 13 |
| Non- | 27 | 7 | 8 | 12 | 13 | 17 |
| seminomatous | | | | | | |
| Total | 52 | 13 | 17 | 12 | 23 | 31 |
| | | (25%) | (32.6%) | (23%) | (44.2%) | (59.6%) |

### b) Serial Measurements of PEP and HCGβ Serum Levels

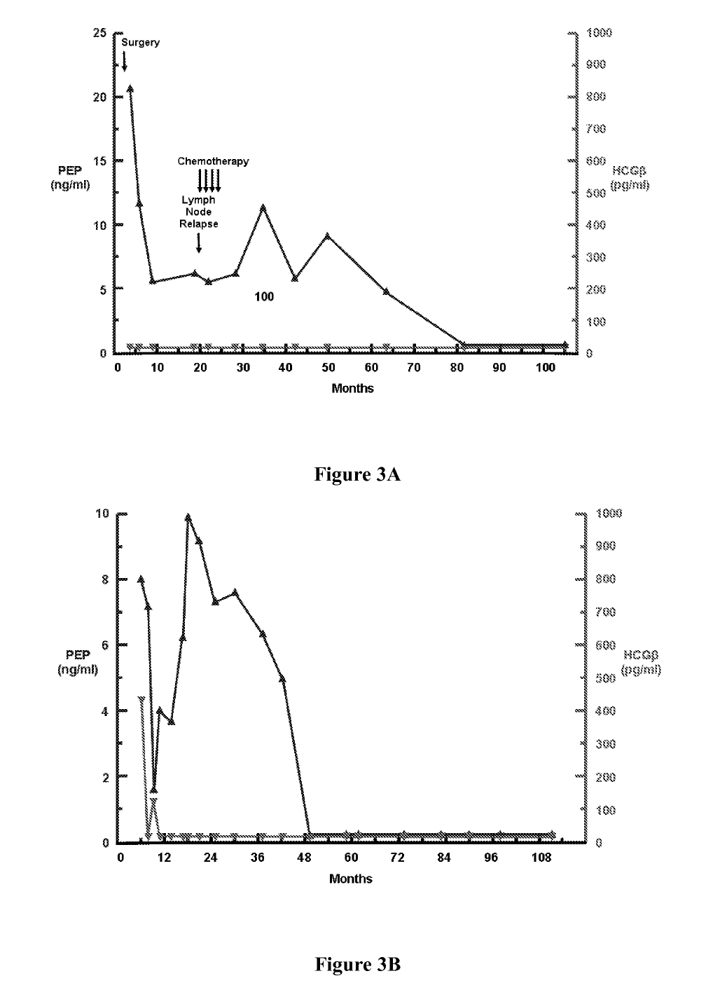

Serial determination of PEP and HCGβ was carried out in two patients followed up for at least four years and taken as representative (FIGS. 3A and 3B). One patient had elevated serum values of PEP for a period of 42 months after orchidectomy, while neither HCGβ nor AFP was ever detected (FIG. 3A). However, in this patient, the initial serum sample available for this study had been drawn 3 months after surgery. It is noteworthy that, after an initial decrease in PEP values following orchidectomy, a rise in serum PEP levels and lymph node relapse occurred concurrently in this patient, who had a mixed form of a non-seminomatous germ cell tumor composed of embryonal carcinoma and seminoma. The other representative patient had measurable levels of PEP, HCGβ and AFP (116 UI/mL) on the initial serum sample drawn prior to orchidectomy. After surgery, AFP levels were consistently below the upper limit of the usual values found in healthy subjects (<5 ng/mL).¹³ A low level of HCGβ (0.122 pg/mL) was still detected 3 months after surgery. In striking contrast, high levels of PEP were detected up to 3 years after surgery for this tumor, which was a non-seminomatous germ cell tumor comprising choriocarcinoma, yolk sac tumor, teratoma and seminoma.

### c) Expression of PEP at the Cellular Level

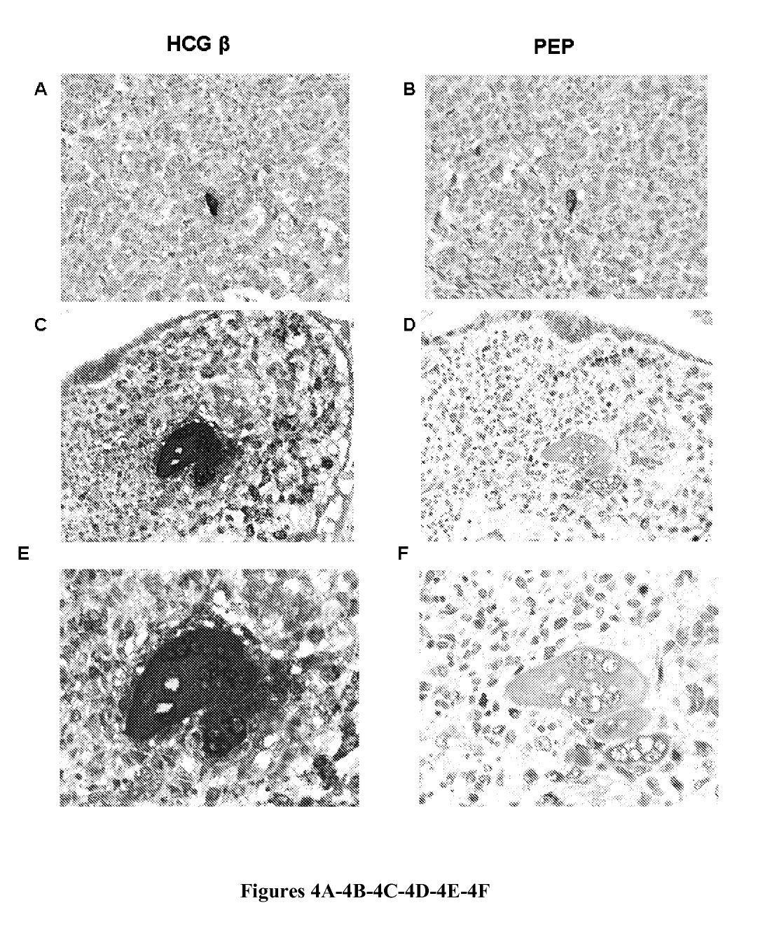

In order to identify PEP-producing cells, immunohistochemistry studies were performed on histological sections of two testis tumors excreting measurable serum levels of PEP. One tumor was a mixed form of non-seminomatous germ cell tumor composed of embryonal carcinoma, teratoma and choriocarcinoma (FIGS. 4A and 4B) and the other was a mixed form of a non-seminomatous germ cell tumor composed of embryonal carcinoma, yolk sac tumor and seminoma with syncytiotrophoblastic cells (FIGS. 4C to 4F). On tissue sections studied, AFP-producing cells were not present. In contrast, HCGβ-producing cells were strongly stained with mAb directed to HCGβ. In these tumors, PEP staining was detected with mAb EPIL¹⁵ in multinucleated (syncytiotrophoblastic) cells, while, in one tumor, staining was also detected in mononucleated cells (FIGS. 4E and 4F). In the latter tumor, the intensity of staining was stronger in mononucleated cells compared to multinucleated cells.

### 3) Conclusion

Testicular cancer is the most common solid tumor in young men between the ages of 15 and 34, and 95% of these cancers are germ-cell tumors, a term that indicates their origin in primordial germ-cells.1 Sensitive tumor markers and effective treatment modalities have transformed the prognosis for these cancers and they are now highly curable. However, several aspects of testicular germ-cell tumors remain challenging: The incidence of these cancers is rising and a delay in diagnosis may still be too long for some patients, thereby affecting their prognosis. Although this prognosis is considered good in a large majority of patients, a group with poor prognosis remains. On a broader perspective, a recently observed slowdown in the decline in mortality rates is cause for concern. Another challenge is the need to minimize long-term toxic effects of therapy without jeopardizing effectiveness. While the three biomarkers, HCG, HCGβ and AFP, effectively contribute to management of testicular cancer, novel biomarkers would be helpful in improving this management. Among the three latter biomarkers, it is noteworthy that, in terms of expression, both HCG and HCGβ share similarities with a distinctive group of antigens called cancer/testis (C/T) antigens. In order for a protein to be designated a C/T antigen, it must be expressed in tumors as well as in testis and/or the placenta, but must not be expressed in more than two non-germ-line normal tissues. When non-germ-line normal tissue expression is detected, this is usually at only a fraction of the level detected in the testis.¹⁴ Like HCGβ, tumor antigen MZ2-E, which was the first C/T antigen described, along with a growing number of C/T antigens (more than 40) which have now been identified, are highly expressed in placenta and tumors.¹⁵ Likewise, PEP is expressed in placenta and tumors: it was reported that c-erbB-2-positive breast cancer cells with high invasion potential express and secrete PEP.¹⁶ Moreover, as expected from a C/T antigen, PEP expression was detected in only one non-germ-line normal tissue and this expression was faint. Thus, PEP expression displays most of the hallmarks of cancer/testis (CT) antigens, and this study was designed to determine whether this antigen is also a biomarker of testicular cancers.

PEP is detected in about half of males presenting with either seminomatous (24%) or non-seminomatous (25.9%) testicular tumors (Table 2). In contrast, measurable serum levels were found in only 2 out of 104 (1.9%) healthy male subjects. It is not uncommon that tumor markers, including HCG and HCGβ, are present in a limited number of healthy male subjects. Indeed, sera from men and non-pregnant women contain low levels of HCG and HCGβ that can be detected by sensitive assays; it is likely that detection of PEP in fewer than 2% of healthy males is related to the sensitivity of the immunoassay used for measuring PEP.^{17,18} More importantly, it was striking that 8 out of 13 (61.5%) PEP-producing tumors did not secrete measurable levels of either HCGβ or AFP. Indeed, in this series of 52 patients with germ-cell testicular cancers, 44.2% of patients had measurable serum levels of HCGβ and/or AFP, while HCGβ, AFP and/or PEP levels were elevated in sera of 59.6% of patients. It is likely that these percentages were underestimated, since most serum samples were collected 1 to 303 days after orchidectomy: in that series, 48.1% of patients with non-seminomatous tumors had elevated serum levels of HCGβ and/or AFP, while it had been previously observed that one or both of these markers are expressed in about three-fourths of non-seminomas.²

Another unexpected observation was the persistence of measurable serum levels of PEP in certain patients for up to 42 months after the end of treatment. It is likely that the persistence of measurable serum levels of PEP indicates the presence of remaining tumor cells. Surgery of residual masses after chemotherapy in patients with testicular cancer show that 45% and 10% of residual masses contain teratomas or active disease, respectively.¹⁹ Moreover, it was reported that patients with a variety of primary germ-cell tumors in the testis and who are treated with radiation therapy and/or chemotherapy in addition to surgery may develop mature teratomas at differing anatomical sites.²⁰ Interestingly, one patient with a non-seminomatous germ-cell tumor composed of embryonal carcinoma and seminoma displayed a rise in PEP serum levels prior to lymph node relapse, followed by persistent serum levels of PEP after completion of chemotherapy (FIG. 3A). Another patient with persistent levels of PEP did not receive radiation therapy or chemotherapy (FIG. 3B). That patient had a non-seminomatous germ-cell tumor composed of choriocarcinoma, yolk sac tumor, teratoma and seminoma. The types of tumor cells that continue to produce PEP thus remain to be determined. Indeed, testicular cancers with a wide variety of histological types secrete PEP. In our series, PEP was excreted by 6 pure seminomas without syncytiotophoblast cells, 1 embryonal carcinoma and 6 mixed tumors containing two or three differing histological types (1 embryonal carcinoma and seminoma, 1 choriocarcinoma and seminoma, 1 teratoma and seminoma, 1 embryonal carcinoma, teratoma and choriocarcinoma, 1 embryonal carcinoma, yolk sac and seminoma with syntytiotrophoblastic cells and 1 choriocarcinoma, yolk sac tumor, teratoma and seminoma). In order to characterize the histological type of expressing cell, immunohistochemistry staining was performed on two mixed tumors with antibodies directed to HCGβ, AFP and PEP: one testis tumor contained embryonal carcinoma, teratoma and choriocarcinoma (FIGS. 4A and 4B) and the other tumor was a mixed form of non-seminomatous germ cell tumor composed of embryonal carcinoma, yolk sac tumor and seminoma with syncytiotrophoblastic cells (FIGS. 4C to 4F). On these tissue sections, multinucleated cells appeared to produce both HCGβ and PEP, with the same (FIGS. 4A and 4B) or differing (FIGS. 4C and 4D) staining intensities. In mononucleated cells, these two proteins may be produced by the same or by distinct mononucleated cells.

Taken together, observations at the serum and cellular levels show that, in contrast to normal placenta in which biosynthesis of HCGβ and PEP may be regulated by common pathways, in germ-cell testicular tumors, their production might be regulated by differing pathways.¹⁰

Finally, PEP is a serum biomarker which provides a complement of information on germ-cell testicular cancers. PEP may be the only biomarker present in sera of seminomatous and non-seminomatous testicular tumors, indicating the presence of tumor cells previously undetected by other serum biomarkers at clinical diagnosis. PEP may also indicate the presence of residual tumor cells still present several years after the end of treatment.

### REFERENCES

1. Bosl G J, Motzer R J: Testicular germ-cell cancer. N Engl J. Med. 337:242-53, 1997.
2. Horwich A, Huddart R, Dearnaley D: Markers and management of germ-cell tumors of the testes. Lancet 352:1535-8, 1998.
3. Levi F, La Vecchia C, Boyle P, et al: Western and eastern European trends in testicular cancer mortality. Lancet 357:1853-4, 2001.
4. Horwich A, Shipley J, Huddart R: Testicular germ-cell cancer. Lancet 367:754-65, 2006.
5. Dieckmann K P: Diagnostic delay in testicular cancer: an analytic chimaera or a worthy goal. Eur Urol 52:1566-8, 2007.
6. Huyghe E, Muller A, Mieusset R, et al: Impact of diagnostic delay in testis cancer: results of a large population-based study. Eur Urol 52:1710-6, 2007.
7. Huyghe E, Matsuda T, Thonneau P: Increasing incidence of testicular cancer worldwide: a review. J. Urol. 170:5-11, 2003.
8. Chassin D, Laurent A, Janneau J L, et al: Cloning of a new member of the insulin gene superfamily (INSL4) expressed in human placenta. Genomics 29:465-70, 1995.
9. Bellet D, Lavaissiere L, Mock P, et al: Identification of pro-EPIL and EPIL peptides translated from insulin-like 4 (INSL4) mRNA in human placenta. J Clin Endocrinol Metab 82:3169-72, 1997.
10. Mock P, Frydman R, Bellet D, et al: Pro-EPIL forms are present in amniotic fluid and maternal serum during normal pregnancy. J Clin Endocrinol Metab 84:2253-6, 1999.
11. Bruni L, Luisi S, Ferretti C, et al: Changes in the maternal serum concentration of proearly placenta insulin-like growth factor peptides in normal vs abnormal pregnancy. Am. J. Obstet. Gynecol. 197:606 e1-4, 2007.
12. Marcillac I, Troalen F, Bidart J M, et al: Free human chorionic gonadotropin beta subunit in gonadal and nongonadal neoplasms. Cancer Res 52:3901-7, 1992.
13. Bellet D H, Wands J R, Isselbacher K J, et al: Serum alpha-fetoprotein levels in human disease: perspective from a highly specific monoclonal radioimmunoassay. Proc Natl Acad Sci USA, 81:3869-73, 1984.
14. Scanlan M J, Simpson A J, Old L J: The cancer/testis genes: review, standardization, and commentary. Cancer Immun. 4:1, 2004.
15. van der Bruggen P, Traversari C, Chomez P, et al: A gene encoding an antigen recognized by cytolytic T lymphocytes on a human melanoma. Science 254:1643-7, 1991.
16. Brandt B, Roetger A, Bidart J M, et al: Early placenta insulin-like growth factor (pro-EPIL) is overexpressed and secreted by c-erbB-2-positive cells with high invasion potential. Cancer Res 62:1020-4, 2002.
17. Stenman U H, Tiitinen A, Alfthan H, et al: The classification, functions and clinical use of different isoforms of HCG. Hum Reprod Update 12:769-84, 2006.
18. Alfthan H, Haglund C, Dabek J, et al: Concentrations of human choriogonadotropin, its beta-subunit, and the core fragment of the beta-subunit in serum and urine of men and nonpregnant women. Clin Chem 38:1981-7, 1992.
19. Flechon A, Rivoire M, Berger N: [Surgery of residual masses after chemotherapy in patients with testicular cancer]. Rev Prat 57:389-98, 2007.
20. Hong W K, Wittes R E, Hajdu S T, et al: The evolution of mature teratoma from malignant testicular tumors. Cancer 40:2987-92, 1977.

1. An in vitro method for detecting and/or classifying a testicular cancer in a subject, comprising a step of determining the expression level of the gene encoding the pro-EPIL peptide in a biological sample isolated from said subject wherein overexpression of said gene encoding the pro-EPIL peptide is indicative of the presence of a testicular cancer and/or class of testicular germ cell tumor.

2. The method of claim 1, wherein said method further comprises a step of determining the expression level of at least one gene selected from the group consisting of the genes encoding the human gonadotropin HCG, its beta subunit HCGβ, and the human alpha-fetoprotein AFP, in said biological sample wherein overexpression of is indicative of the presence of a testicular cancer and/or class of testicular germ cell tumor.

3. The method of claim 1, wherein said method further comprises a step of determining the expression level of the gene encoding the HCG, or its subunit HCGβ, and of the gene encoding the human AFP in said biological sample, wherein overexpression of at least one of said genes is indicative of the presence of a testicular cancer and/or class of testicular germ cell tumor.

4. The method of claim 1, wherein said testicular cancer and/or class of testicular germ cell tumor is seminomatous.

5. The method of claim 1, wherein the presence of an overexpression of the gene encoding the pro-EPIL peptide, an overexpression of the gene encoding the HCGβ, and an overexpression of the gene encoding the alpha-fetoprotein AFP in said biological sample is indicative of the presence of a non-seminomatous testicular cancer and/or class of testicular germ cell tumor.

6. A method for identifying a candidate compound for a pharmacological agent useful in the treatment of testicular cancer comprising the steps of:
a) contacting a non-human mammal subject presenting a testicular cancer with a candidate pharmacological agent; and
b) determining the expression level of the gene encoding the pro-EPIL peptide in a biological sample isolated from said subject,
wherein a decrease in the expression level determined in step b) indicates that the candidate pharmacological agent is a candidate compound for a pharmacological agent useful in the treatment of testicular cancer.

7. A method for evaluating the effect in a subject of a treatment for testicular cancer, particularly a comprising the steps of:
a) determining the expression level of the gene encoding the pro-EPIL peptide in a biological sample isolated from said subject;
b) determining a second expression level of the gene encoding the pro-EPIL peptide in a biological sample isolated from said subject; and
c) comparing the first and second amounts of expression level of the gene encoding the pro-EPIL peptide in said biological samples,
wherein a decrease of the expression level determined in step b) indicates the regression of said testicular cancer.

8. The method of claim 1, wherein the expression level is determined by detecting the presence, absence, or level of a molecule selected from the group consisting of mRNA transcribed from said gene, a polypeptide encoded by said gene, and specific fragments fragment thereof.

9. The method of claim 8, wherein said polypeptide is detected or quantified by a method selected from the group consisting of western blot analysis, chromatography, immunoassay, and immunohistochemistry.

10. The method of claim 1, wherein the biological sample is selected from the group consisting of whole blood, blood serum, plasma, tumor biopsy, and combinations thereof.

11. The method of claim 9, wherein said immunoassay is selected from the group consisting of ELISA immunoassay and radioimmunoassay in blood serum.

12. The method of claim 9, wherein said polypeptide is detected or quantified by immunohistochemistry at the cellular level.

13. A kit comprising:
a) an antibody directed specifically against the pro-EPIL peptide; and
b) an antibody directed specifically against at least one polypeptide selected from the group consisting of the HCG, its subunit HCGβ, and the human AFP polypeptides.

14. The kit of claim 13, comprising:
a) an antibody directed specifically against the pro-EPIL peptide;
b) an antibody directed specifically against a polypeptide selected from the group consisting of the HCG and its subunit HCGβ; and
c) an antibody directed specifically against the human AFP polypeptide.

15. A kit comprising:
a) a set of primers capable of amplifying specifically the Pro-EPIL RNA; and
b) a set of primers capable of amplifying specifically the RNA from the group consisting of the HCG, its subunit HCGβ, and the human alpha-fetoprotein AFP RNA.

16. The kit of claim 15, wherein:
b) is composed of a set of primers capable of amplifying specifically the HCG or its subunit HCGβ RNA and
a set of primers capable of amplifying specifically the human AFP RNA.

17. A kit comprising:
a) a nucleic probe capable of hybridizing specifically with the Pro-EPIL RNA; and
b) a nucleic probe capable of hybridizing specifically with at least one RNA selected from the group consisting of the human gonadotropin HCG, its beta subunit HCGβ, and the human alpha-fetoprotein AFP RNA.

18. The kit of claim 17, comprising:
a) a nucleic probe capable of hybridizing specifically with the Pro-EPIL RNA;
b) a nucleic probe capable of hybridizing specifically with the RNA selected from the group consisting of HCG and its subunit HCGβ RNA; and
c) a nucleic probe capable of hybridizing specifically with the human AFP RNA.

19. The kit of claim 13, suitable for performing the method according to claim 1.

20. A solid-phase nucleic acid molecule array comprising:
a) a nucleic acid molecule capable of hybridizing specifically with the pro-EPIL RNA; and
b) a nucleic acid molecule capable of hybridizing specifically with at least one RNA selected from the group consisting of the human gonadotropin HCG, its beta subunit HCGβ, and the human alpha-fetoprotein AFP RNA.

21. A solid-phase protein microarray comprising:
a) an antibody directed specifically against the pro-EPIL peptide; and
b) an antibody directed specifically against at least one polypeptide selected from the group consisting of the HCG, its subunit HCGβ, and the human alpha-fetoprotein AFP polypeptide, fixed to a solid substrate.

22. A method for diagnosing testicular cancer wherein the expression of the pro-EPIL gene is a biomarker of said testicular cancer.

23. The method of claim 1, wherein said testicular cancer and/or class of testicular germ cell tumor is non-seminomatous.

24. The method of claim 6, wherein said method further comprises:
c) determining the expression level of at least one gene selected from the group consisting of the genes encoding the HCG, its subunit HCGβ, and the human AFP in said sample.

25. The method according to claim 6, wherein said method further comprises:
c) determining the expression level of at least one gene selected from the group consisting of the genes encoding the HCG, its subunit HCGβ, and the human AFP, in said sample and
wherein said expression level determined in step c) decreases.

26. The method of claim 6, wherein the testicular cancer is a testicular germ cell tumor.

27. The method of claim 7, wherein said method is for evaluating the effect in a subject of a treatment for a testicular germ cell tumor.

28. The method of claim 6, wherein the expression level is determined by detecting the presence, absence or level of a molecule selected from the group consisting of mRNA transcribed from said gene, polypeptide encoded by said gene, and specific fragments thereof.

29. The method of claim 7, wherein the expression level is determined by detecting the presence, absence or level of a molecule selected from the group consisting of mRNA transcribed from said gene, polypeptide encoded by said gene, and specific fragments thereof.

30. The method of claim 28, wherein said polypeptide is detected or quantified by a method selected from the group consisting of western blot analysis, chromatography, immunoassay, and immunohistochemistry.

31. The method of claim 29, wherein said polypeptide is detected or quantified by a method selected from the group consisting of western blot analysis, chromatography, immunoassay, and immunohistochemistry.

32. The method of claim 6, wherein said biological sample is selected from the group consisting of whole blood, blood serum, plasma, tumor biopsy, and combinations thereof.

33. The method of claim 7, wherein said biological sample is selected from the group consisting of whole blood, blood serum, plasma, tumor biopsy, and combinations thereof.

34. The method of claim 9, wherein said immunoassay is selected from the group consisting of ELISA immunoassay and radioimmunoassay in plasma.

35. The method of claim 30, wherein said immunoassay is selected from the group consisting of ELISA immunoassay and radioimmunoassay in blood serum.

36. The method of claim 30, wherein said immunoassay is selected from the group consisting of ELISA immunoassay and radioimmunoassay in plasma.

37. The method of claim 31, wherein said immunoassay is selected from the group consisting of ELISA immunoassay and radioimmunoassay in blood serum.

38. The method of claim 31, wherein said immunoassay is selected from the group consisting of ELISA immunoassay and radioimmunoassay in plasma.

39. The method of claim 30, wherein said polypeptide is detected or quantified by immunohistochemistry at the cellular level.

40. The method of claim 31, wherein said polypeptide is detected or quantified by immunohistochemistry at the cellular level.

41. The method of claim 12, wherein said cellular level is selected from the group of cells consisting of multinucleated cells, mononucleated cells, and combinations thereof.

42. The method of claim 39, wherein said cellular level is selected from the group of cells consisting of multinucleated cells, mononucleated cells, and combinations thereof.

43. The method of claim 40, wherein said cellular level is selected from the group of cells consisting of multinucleated cells, mononucleated cells, and combinations thereof.

44. A kit comprising:
a) a set of primers capable of amplifying specifically the Pro-EPIL cDNA; and
b) a set of primers capable of amplifying specifically the cDNA from the group consisting of the HCG, its subunit HCGβ, and the human alpha-fetoprotein AFP cDNA.

45. A kit comprising:
a) a set of primers capable of amplifying specifically the Pro-EPIL RNA; and
b) a set of primers capable of amplifying specifically the cDNA from the group consisting of the HCG, its subunit HCGβ, and the human alpha-fetoprotein AFP cDNA.

46. A kit comprising:
a) a set of primers capable of amplifying specifically the Pro-EPIL cDNA; and
b) a set of primers capable of amplifying specifically the RNA from the group consisting of the HCG, its subunit HCGβ, and the human alpha-fetoprotein AFP RNA.

47. The kit of claim 44, wherein
b) is composed of a set of primers capable of amplifying specifically the HCG or its subunit HCGβ cDNA and a set of primers capable of amplifying specifically the human AFP cDNA.

48. The kit of claim 45, wherein
b) is composed of a set of primers capable of amplifying specifically the HCG or its subunit HCGβ cDNA and a set of primers capable of amplifying specifically the human AFP cDNA.

49. The kit of claim 46, wherein
b) is composed of a set of primers capable of amplifying specifically the HCG or its subunit HCGβ RNA and a set of primers capable of amplifying specifically the human AFP RNA.

50. The kit of claim 13, suitable for performing the method according to claim 6.

51. The kit of claim 13, suitable for performing the method according to claim 7.

52. The kit of claim 15, suitable for performing the method according to claim 1.

53. The kit of claim 15, suitable for performing the method according to claim 6.

54. The kit of claim 15, suitable for performing the method according to claim 7.

55. The kit of claim 17, suitable for performing the method according to claim 1.

56. The kit of claim 17, suitable for performing the method according to claim 6.

57. The kit of claim 17, suitable for performing the method according to claim 7.

58. The method of claim 22, wherein said testicular cancer is a testicular germ cell tumor.


SEQUENCE LISTING (11 sequences)

SEQ ID NO: 1 - protein, 139 aa, Homo sapiens
MASLFRSYLPAIWLLLSQLLRESLAAELRGCGPRFGKHLLSYCPMPEKTFTTTPGGWLLESGRPKEMVSTSNNKDGQALGTTSEFIPNLSPELKKPLSEGQPSLKKIILSRKKRSGRHRFDPFCCEVICDDGTSVKLCT

SEQ ID NO: 2 - protein, 22 aa, Homo sapiens
MASLFRSYLPAIWLLLSQLLRE

SEQ ID NO: 3 - protein, 30 aa, Homo sapiens
SLAAELRGCGPRFGKHLLSYCPMPEKTFTT

SEQ ID NO: 4 - protein, 50 aa, Homo sapiens
LESGRPKEMVSTSNNKDGQALGTTSEFIPNLSPELKKPLSEGQPSLKKII

SEQ ID NO: 5 - protein, 25 aa, Homo sapiens
SGRHRFDPFCCEVICDDGTSVKLCT

SEQ ID NO: 6 - DNA, 617 bp, Homo sapiens
agtctggagcccagaagggacacaccagcacagtctggtaggctacagcagcaagtctctaaagaaaggctgagaacacccagaacaggagagttcaggtccaggatggccagcctgttccggtcctatctgccagcaatctggctgctgctgagccaactccttagagaaagcctagcagcagagctgaggggatgtggtccccgatttggaaaacacttgctgtcatattgccccatgcctgagaagacattcaccaccaccccaggagggtggctgctggaatctggacgtcccaaagaaatggtgtcaacctccaacaacaaagatggacaagccttaggtacgacatcagaattcattcctaatttgtcaccagagctgaagaaaccactgtctgaagggcagccatcattgaagaaaataatactttcccgcaaaaagagaagtggacgtcacagatttgatccattctgttgtgaagtaatttgtgacgatggaacttcagttaaattatgtacatagtagagtaatcatggactggacatctcatccattctcatatgtattctcaatgacaaattcactgatgcccaattaaatgattgctgtttaaa

SEQ ID NO: 7 - DNA, 417 bp, Homo sapiens
atggccagcctgttccggtcctatctgccagcaatctggctgctgctgagccaactccttagagaaagcctagcagcagagctgaggggatgtggtccccgatttggaaaacacttgctgtcatattgccccatgcctgagaagacattcaccaccaccccaggagggtggctgctggaatctggacgtcccaaagaaatggtgtcaacctccaacaacaaagatggacaagccttaggtacgacatcagaattcattcctaatttgtcaccagagctgaagaaaccactgtctgaagggcagccatcattgaagaaaataatactttcccgcaaaaagagaagtggacgtcacagatttgatccattctgttgtgaagtaatttgtgacgatggaacttcagttaaattatgtaca

SEQ ID NO: 8 - DNA, 66 bp, Homo sapiens
atggccagcctgttccggtcctatctgccagcaatctggctgctgctgagccaactccttagagaa

SEQ ID NO: 9 - DNA, 90 bp, Homo sapiens
agcctagcagcagagctgaggggatgtggtccccgatttggaaaacacttgctgtcatattgccccatgcctgagaagacattcaccacc

SEQ ID NO: 10 - DNA, 150 bp, Homo sapiens
ctggaatctggacgtcccaaagaaatggtgtcaacctccaacaacaaagatggacaagccttaggtacgacatcagaattcattcctaatttgtcaccagagctgaagaaaccactgtctgaagggcagccatcattgaagaaaataata

SEQ ID NO: 11 - DNA, 75 bp, Homo sapiens
agtggacgtcacagatttgatccattctgttgtgaagtaatttgtgacgatggaacttcagttaaattatgtaca